(19) United States
(12) Patent Application Publication
(10) Pub. No.: US 2008/0183245 A1
(43) Pub. Date: Jul. 31, 2008

(54) TELEMETRY OF EXTERNAL PHYSIOLOGICAL SENSOR DATA AND IMPLANTABLE MEDICAL DEVICE DATA TO A CENTRAL PROCESSING SYSTEM

(76) Inventors: Geeske Van Oort, Velp (NL); Willem Boute, Brummen (NL); Gustaaf A.P. Stoop, Dieren (NL)

Correspondence Address: MEDTRONIC, INC., 710 MEDTRONIC PARKWAY NE, MINNEAPOLIS, MN, 55432-9924, US

(21) Appl. No.: 11/669,350

(22) Filed: Jan. 31, 2007

(51) Int. Cl. A61N 1/00

(52) U.S. Cl. 607/60

(57) ABSTRACT

An implantable medical device (IMD) system as disclosed herein includes a telemetry transceiver device that can receive wireless IMD data from an IMD, along with wireless physiological sensor data from an external physiological sensor device. Wireless communication between the IMD and the telemetry transceiver device is performed in accordance with a first wireless data communication protocol, and wireless communication between the external physiological sensor device and the telemetry transceiver device is performed in accordance with a second wireless data communication protocol. The telemetry transceiver device can function as a hub that communicates patient data to a centralized remote computing architecture. The remote computing architecture may include software applications for processing the received patient data.

### TECHNICAL FIELD

Embodiments of the subject matter described herein relate generally to implantable medical device (IMD) systems. More particularly, the subject matter described herein relates to a system for collecting and communicating wireless IMD data along with data generated by external physiological sensor devices.

### BACKGROUND

IMDs are used to treat patients suffering from a variety of conditions.

IMDs can be utilized in a variety of applications, such as drug or fluid delivery, patient monitoring, and therapeutic uses for areas of medicine such as cardiology, endocrinology, hematology, neurology, muscular disorders, gastroenterology, urology, ophthalmology, otolaryngology, orthopedics, and similar medical subspecialties. Many IMDs are designed to generate and deliver electrical pulses to stimulate body tissue, muscles, nerves, brain cells, body fluid, etc.

Examples of IMDs involving cardiac devices are implantable pacemakers and implantable cardioverter-defibrillators. Such electronic medical devices generally monitor the electrical activity of the heart and provide electrical stimulation to one or more of the heart chambers when necessary. For example, pacemakers are designed to sense arrhythmias, i.e., disturbances in heart rhythm, and, in turn, provide appropriate electrical stimulation pulses at a controlled rate to selected chambers of the heart in order to correct the arrhythmias and restore the proper heart rhythm. The types of arrhythmias that may be detected and corrected by such IMDs include bradycardias (unusually slow heart rates) and certain tachycardias (unusually fast heart rates).

Existing IMDs are often capable of supporting telemetry communication with an external programmer or an interface device that can transfer IMD data to a computer-based diagnostic or monitoring application. For example, the CARELINK® branded system and service offered by Medtronic is an Internet-based remote monitoring service for patients with compatible IMDs. This service enables secure patient monitoring by a remote caregiver. In practice, however, certain types of physiological patient data will not be available from the IMD. For example, an IMD used for cardiac monitoring may be designed to only provide information related to heart rate, myocardial waveforms, and the like. Additional physiological data, such as blood pressure and body temperature, might not be readily available from a cardiac monitoring IMD, although these might be important to the caregiver for clinical decision making.

### BRIEF SUMMARY

A medical device system as described herein collects IMD data and external physiological sensor data in a remote storage element for processing by an appropriate diagnostic or monitoring application. Information that can easily be derived or obtained from external sensor devices include, for example, blood pressure, body temperature, body weight, body fat ratio, body fluid ratio, blood glucose levels, and the like. The external physiological sensor devices that measure these parameters can utilize wireless telemetry techniques to transmit their measured results to either the IMD (for later transmission together with the IMD data) or to a telemetry transceiver device that is also utilized to collect the IMD data directly from the IMD, either using the same or an alternative telemetry system as the IMD. Alternative telemetry systems include commercially available systems such as those having Bluetooth wireless data communication features. In turn, the telemetry transceiver device can forward the collected data to a remote computing architecture for storage and/or processing.

The above and other aspects may be carried out in one embodiment by a system for handling medical device data. The system includes: an external physiological sensor device having wireless data communication capabilities; a telemetry transceiver device configured to wirelessly receive IMD data from an IMD, and configured to obtain physiological sensor data generated by the external physiological sensor device; a communication module for the telemetry transceiver device, the communication module being configured to send patient data that conveys the IMD data and the physiological sensor data; and a remote computing architecture in data communication with the communication module, the remote computing architecture being configured to receive the patient data from the communication module.

The above and other aspects may be carried out in one embodiment by a telemetry transceiver device for handling medical device data. The telemetry transceiver device includes: an IMD communication module configured to wirelessly receive IMD data from an IMD; an external device communication module configured to receive physiological sensor data from an external physiological sensor device; and a remote communication module coupled to the IMD communication module and coupled to the external device communication module. The remote communication module is configured to send patient data to a remote computing architecture, wherein the patient data conveys the IMD data and the physiological sensor data. Such a module may be connected to the computing architecture using wired technology or wireless technology (for example, cellular-like technology).

The above and other aspects may be carried out in one embodiment by a method for handling medical device data. The method involves: wirelessly receiving IMD data at a telemetry transceiver device, the IMD data originating at an IMD; obtaining (by the telemetry transceiver device) physiological sensor data generated by an external physiological sensor device; generating (by the telemetry transceiver device) patient data that conveys the IMD data and the physiological sensor data; and sending (by the telemetry transceiver device) the patient data to a remote computing architecture.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a schematic representation of an embodiment of a system for handling medical device data;

FIG. 2 is a schematic representation of another embodiment of a system for handling medical device data;

FIG. 3 is a schematic representation of an embodiment of an IMD suitable for use in the systems shown in FIG. 1 and FIG. 2;

FIG. 4 is a schematic representation of an embodiment of a telemetry transceiver device suitable for use in the systems shown in FIG. 1 and FIG. 2; and

FIG. 5 is a flow chart that illustrates an embodiment of a medical device data telemetry process.

### DETAILED DESCRIPTION

The following detailed description is merely illustrative in nature and is not intended to limit the embodiments of the invention or the application and uses of such embodiments. Furthermore, there is no intention to be bound by any expressed or implied theory presented in the preceding technical field, background, brief summary or the following detailed description.

Techniques and technologies may be described herein in terms of functional and/or logical block components and various processing steps. It should be appreciated that such block components may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments may be practiced in conjunction with any number of data transmission protocols and that the system described herein is merely one suitable example.

For the sake of brevity, conventional techniques and features related to IMDs, wireless transceivers, data communication protocols, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in a practical embodiment.

The following description refers to elements or nodes or features being “connected” or “coupled” together. As used herein, unless expressly stated otherwise, “connected” means that one element/node/feature is directly joined to (or directly communicates with) another element/node/feature, and not necessarily mechanically. Likewise, unless expressly stated otherwise, “coupled” means that one element/node/feature is directly or indirectly joined to (or directly or indirectly communicates with) another element/node/feature, and not necessarily mechanically.

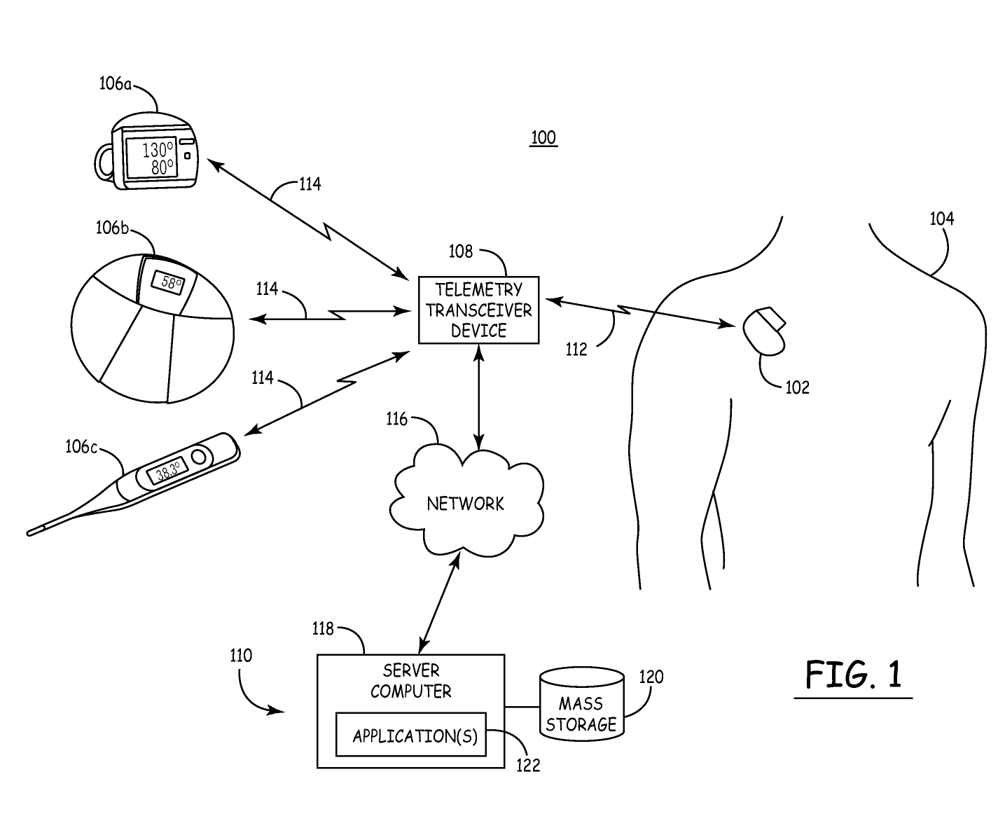

FIG. 1 is a schematic representation of an embodiment of a system 100 for handling medical device data. System 100 generally includes at least one IMD 102 (for simplicity, only one IMD 102 is depicted in FIG. 1) implanted within the body of a patient 104, at least one external physiological sensor device 106, a telemetry transceiver device 108, and a remote computing architecture 110. For this example, system 100 includes one IMD 102 and three external physiological sensor devices 106. In practice, however, system 100 may include one, two, or more than three external physiological sensor devices 106. Moreover, system 100 may be configured to support more than one IMD 102 (where the total number of supported IMDs may all be implanted within the same patient 104 or within more than one patient). Although only one telemetry transceiver device 108 is shown in FIG. 1, a practical system may include more than one, for example, one “stationary” wired version for use at home and one mobile version for use when the patient 104 leaves the house.

The embodiments described herein can be implemented using any IMD that is configured to communicate in a wireless manner using telemetry techniques. An IMD suitable for use with the systems described herein may function as a monitoring device, as a therapeutic device, or a combination thereof. For example, the IMD may deliver electrical pulses as stimulation or therapy to body tissue, fluid, muscle, bone, etc., and/or it may be configured to receive sensor data that is indicative of one or more physiological processes. Such IMDs include pacemakers as well as ICDs, drug delivery pumps, cardiomyostimulators, cardiac and other physiologic monitors, nerve and muscle stimulators, deep brain stimulators, cochlear implants, and artificial organs (e.g., artificial hearts). In addition, as the technology advances, it is contemplated that IMDs shall become even more complex with respect to programmable operating modes, menus of operating parameters, and monitoring capabilities of increasing varieties of physiologic conditions and electrical signals. It is to be appreciated that embodiments of the subject matter described herein will be applicable in such emerging IMD technology as well.

As described in more detail below, IMD 102 is suitably configured to generate and wirelessly transmit IMD data to telemetry transceiver device 108. IMD data may include, without limitation: physiological patient data collected by implanted sensors that communicate with, are coupled to, or are integrated in IMD 102; status information related to the operation, condition, state, or characteristics of IMD 102 and/or sensors or devices coupled to IMD 102; alarms, alerts, or reminders; IMD battery status information; sensor status information; or the like. In certain embodiments, IMD 102 can also wirelessly receive data from telemetry transceiver device 108, where such data may include, without limitation: physiological patient data obtained by one or more of the external physiological sensor devices 106; command, control, or programming instructions (which may originate at remote computing architecture 110); or the like.

IMD 102 can be suitably configured to communicate with telemetry transceiver device 108 in accordance with a desired wireless data communication protocol. In one preferred embodiment, IMD 102 communicates with telemetry transceiver device 108 using a wireless telemetry protocol capable of communication over a relatively long distance (e.g., up to 10 meters), allowing the patient to move relative to the location of transceiver device 108. This telemetry type is often referred to as long range telemetry. Alternatively or additionally, IMD 102 may be configured to communicate with telemetry transceiver device 108 in a manner that is compliant with one or more of the following wireless data communication protocols (without limitation): an IrDA protocol, a Bluetooth protocol, an IEEE 802.15 protocol, an IEEE 802.11 protocol, an IEEE 802.16 protocol, a direct sequence spread spectrum protocol; a frequency hopping spread spectrum protocol, a cellular telephony protocol, a cordless telephony protocol, a wireless home network communication protocol, a paging network protocol, a magnetic induction protocol, a wireless medical telemetry service (WMTS) protocol, an industrial, scientific, and medical (ISM) protocol, a GPRS protocol, and a wireless USB protocol.

An external physiological sensor device 106 may be, without limitation: a blood pressure measurement device, a body weight measurement device, a body fat measurement device, a body temperature measurement device, a blood glucose measurement device, a heart rate measurement device, a lung capacity measurement device, or any combination thereof. For this example, FIG. 1 depicts a blood pressure measurement device 106a, a scale 106b configured to measure body weight (and/or to measure body fat), and a thermometer 106c configured to measure body temperature.

Each external physiological sensor device 106 preferably has wireless data communication capabilities that support wireless data communication with telemetry transceiver device 108. Alternatively or additionally, any external physiological sensor device 106 may support data communication with telemetry transceiver device 108 using a physical data communication link such as a USB cable, an Ethernet cable, or the like. External physiological sensor devices 106 are suitably configured to generate and wirelessly transmit physiological sensor data to telemetry transceiver device 108. In this regard, physiological sensor data may include, without limitation: the physiological patient data measured by external physiological sensor devices 106; status information related to the operation, condition, state, or characteristics of external physiological sensor devices 106 and/or sensors or devices coupled to external physiological sensor devices 106; alarms, alerts, or reminders; patient queries; or the like. In certain embodiments, an external physiological sensor device 106 can also wirelessly receive data from telemetry transceiver device 108, where such data may include, without limitation: command, control, or programming instructions (which may originate at remote computing architecture 110); status information related to the patient's condition; status information related to the operation, condition, state, or characteristics of IMD 102, sensors or devices coupled to IMD 102, telemetry transceiver device 108, and/or sensors or devices coupled to telemetry transceiver device 108; alarms, alerts, or reminders; patient queries; or the like.

An external physiological sensor device 106 can be suitably configured to communicate with telemetry transceiver device 108 in accordance with a desired wireless data communication protocol. In one preferred embodiment, external physiological sensor devices 106 communicate with telemetry transceiver device 108 using a long range wireless telemetry protocol. Alternatively or additionally, external physiological sensor devices 106 may be configured to communicate with telemetry transceiver device 108 in a manner that is compliant with one or more of the following wireless data communication protocols (without limitation): an IrDA protocol, a Bluetooth protocol, an IEEE 802.15 protocol, an IEEE 802.11 protocol, an IEEE 802.16 protocol, a direct sequence spread spectrum protocol; a frequency hopping spread spectrum protocol, a cellular telephony protocol, a cordless telephony protocol, a wireless home network communication protocol, a paging network protocol, a magnetic induction protocol, a wireless medical telemetry service (WMTS) protocol, an industrial, scientific, and medical (ISM) protocol, a GPRS protocol, and a wireless USB protocol.

Telemetry transceiver device 108 is suitably configured to wirelessly receive IMD data from IMD 102 via at least one wireless link 112. Telemetry transceiver device 108 is also configured to obtain physiological sensor data generated by an external physiological sensor device 106. In the embodiment depicted in FIG. 1, telemetry transceiver device 108 receives the physiological sensor data from external physiological sensor devices 106, preferably via respective wireless links 114. Moreover, telemetry transceiver device 108 may be configured to wirelessly forward the physiological sensor data to IMD 102. In one practical implementation, telemetry transceiver device 108 wirelessly receives the IMD data from IMD 102 in accordance with a particular wireless data communication protocol (for example, a far field telemetry protocol), and wirelessly receives the physiological sensor data from external physiological sensor devices 106 in accordance with a different wireless data communication protocol (for example, a Bluetooth protocol). In another practical embodiment, telemetry transceiver device 108 utilizes the same wireless data communication protocol for data transfer between IMD 102 and external physiological sensor devices 106. Data from external physiological sensor devices 106 and from IMD 102 can be time stamped to synchronize the two or more data sets. Alternatively, any suitable methodology can be utilized to achieve data synchronization.

As described in more detail below, telemetry transceiver device 108 may include or cooperate with a suitably configured communication module that sends patient data to remote computing architecture 110. For the illustrated example, this communication module is integrated into telemetry transceiver device 108, and the integrated communication module is suitably configured to send patient data that conveys the IMD data and the physiological sensor data as needed. The patient data can be arranged and formatted as desired to accommodate the particular data communication protocol and technologies utilized by telemetry transceiver device 108.

Telemetry transceiver device 108 is preferably configured to support data communication over a network 116. In practice, network 116 may include, without limitation: a local area network; a wide area network; the Internet; a cellular network; a mobile telephone network; or the like. In this regard, telemetry transceiver device 108 may be implemented as a mobile telephone (e.g., a cellular telephone, smart phone, PDA, or the like), and its communication module may include a mobile telephone transceiver that is configured to communicate the patient data via a mobile telephone network. Alternatively, telemetry transceiver device 108 may be implemented as any computing device (e.g., a desktop computer, a portable computer, or the like), and its communication module may include a wired or wireless network interface that is configured to communicate the patient data via a wired or wireless network. Depending upon its implementation, the network communication module of telemetry transceiver device 108 may support one or more of the wireless data communication protocols mentioned above. Moreover, the network communication module of telemetry transceiver device 108 may be configured to support one or more wire or cable based data communication protocols, techniques, or methodologies, including, without limitation: Ethernet; home network communication protocols; USB; IEEE 1394 (Firewire); hospital network communication protocols; and proprietary data communication protocols. In an embodiment, telemetry transceiver device 108 may utilize a wired/cabled device interface, which may include or be realized as hardware, software, and/or firmware, such as a suitably configured and formatted port, connector, jack, plug, receptacle, socket, adaptor, or the like.

Remote computing architecture 110 may be realized with any number of hardware, software, and/or firmware components. For example, remote computing architecture 110 may include a server computer 118 and a suitable amount of mass storage 120. In one practical implementation, server computer 118 includes one or more software applications 122, which may be written to support multiple patients and multiple systems. Such applications 122 may include, without limitation: a patient monitoring application; a diagnostic application; an IMD programming application; an application that controls the operation of IMD 102; an application that controls the operation of external physiological sensor devices 106; a management application for telemetry transceiver device 108; an expert system for interpretation of the combined IMD and external sensor data (which may include a clinical decision support system); or the like.

Remote computing architecture 110 is in data communication with telemetry transceiver device 108. For this embodiment, remote computing architecture 110 communicates with telemetry transceiver device 108 via network 116. Remote computing architecture 110 is generally configured to receive the patient data from the communication module of telemetry transceiver device 108. Remote computing architecture 110 can store and/or process the received patient data in an appropriate manner. For example, remote computing architecture 110 may generate, in response to the patient data, control instructions for an external physiological sensor device 106, and send the control instructions to that external physiological sensor device 106 via telemetry transceiver device 108. As another example, the remote computing architecture may generate, in response to the patient data, patient or device status information (e.g., patient monitoring information, an alarm indication, or the like) for an external physiological sensor device 106, and send the status information to that external physiological sensor device 106 via telemetry transceiver device 108. As yet another example, remote computing architecture 110 may generate, in response to the patient data, control instructions for IMD 102, and send the control instructions to IMD 102 via telemetry transceiver device 108, e.g., to carry out additional measurements in an attempt to confirm or reject certain conclusions from an expert system located in the remote architecture.

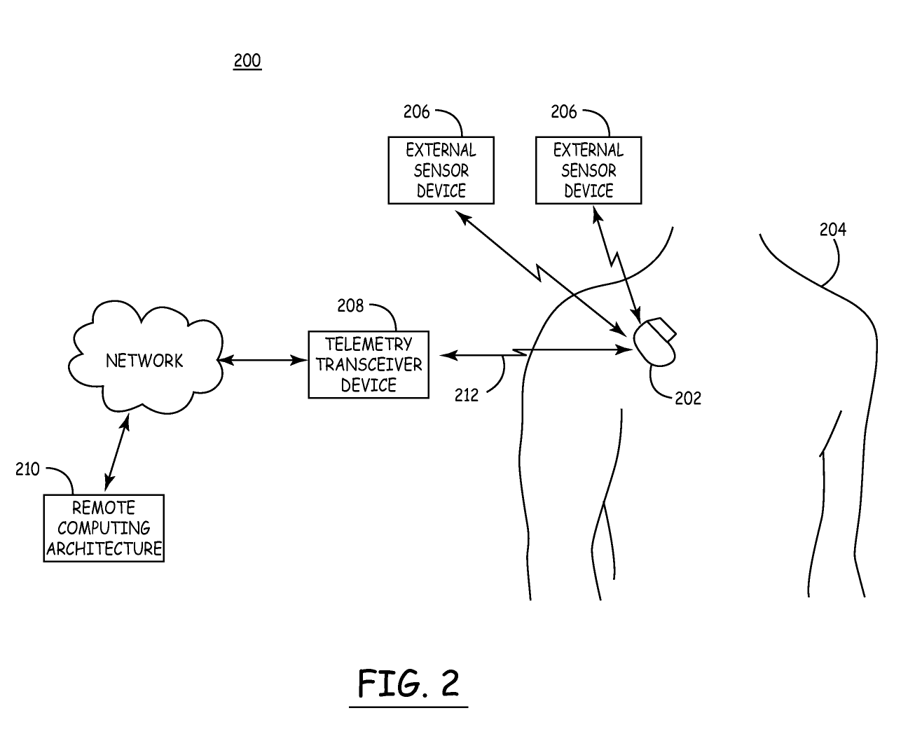

FIG. 2 is a schematic representation of another embodiment of a system 200 for handling medical device data. System 200 generally includes at least one IMD 202 implanted within the body of a patient 204, at least one external physiological sensor device 206, a telemetry transceiver device 208, and a remote computing architecture 210. System 200 shares some common components, features, and traits with system 100. For the sake of brevity, such common features, functions, and elements will not be redundantly described in the context of system 200.

IMD 202 is suitably configured to generate and wirelessly transmit IMD data to telemetry transceiver device 208. In certain embodiments, IMD 202 can also wirelessly receive data from telemetry transceiver device 208, where such data may include, without limitation: command, control, or programming instructions (which may originate at remote computing architecture 210) for IMD 202; command, control, or programming instructions (which may originate at remote computing architecture 210) for external physiological sensor device 206; or the like. In addition, IMD 202 is suitably configured to wirelessly communicate with external physiological sensor devices 206. Accordingly, IMD 202 may require additional processing logic and/or an additional wireless communication module to enable it to cooperate with external physiological sensor devices 206.

For system 200, each external physiological sensor device 206 preferably has wireless data communication capabilities that support wireless data communication with IMD 202. Notably, external physiological sensor devices 206 need not support data communication directly with telemetry transceiver device 208. External physiological sensor devices 206 are suitably configured to generate and wirelessly transmit physiological sensor data to IMD 202. In certain embodiments, an external physiological sensor device 206 can also wirelessly receive data from IMD 202, where such data may include, without limitation: command, control, or programming instructions (which may originate at remote computing architecture 210); status information related to the patient's condition; status information related to the operation, condition, state, or characteristics of IMD 202, sensors or devices coupled to IMD 202, telemetry transceiver device 208, and/or sensors or devices coupled to telemetry transceiver device 208; alarms, alerts, or reminders; or the like.

An external physiological sensor device 206 can be suitably configured to communicate with IMD 202 in accordance with a desired wireless data communication protocol. In one preferred embodiment, external physiological sensor devices 206 communicate with IMD 202 using a long range wireless telemetry protocol. Alternatively or additionally, external physiological sensor devices 206 may be configured to communicate with IMD 202 in a manner that is compliant with one or more of the following wireless data communication protocols (without limitation): an IrDA protocol, a Bluetooth protocol, an IEEE 802.15 protocol, an IEEE 802.11 protocol, an IEEE 802.16 protocol, a direct sequence spread spectrum protocol; a frequency hopping spread spectrum protocol, a cellular telephony protocol, a cordless telephony protocol, a wireless home network communication protocol, a paging network protocol, a magnetic induction protocol, a wireless medical telemetry service (WMTS) protocol, an industrial, scientific, and medical (ISM) protocol, a GPRS protocol, and a wireless USB protocol.

Telemetry transceiver device 208 is suitably configured to wirelessly receive IMD data from IMD 202 via at least one wireless link 212. Telemetry transceiver device 208 is also configured to obtain physiological sensor data generated by external physiological sensor devices 206. In the embodiment depicted in FIG. 2, telemetry transceiver device 208 wirelessly receives the physiological sensor data from IMD 202, preferably via wireless link 212. In this regard, IMD 202 can serve as a wireless repeater device that forwards the physiological sensor data to telemetry transceiver device 208. In one practical implementation, the same wireless data communication protocol (for example, a long range telemetry protocol), is utilized for data transfer between external physiological sensor devices 206 and IMD 202, and for data transfer between IMD 202 and telemetry transceiver device 208. In another practical embodiment, one wireless data communication protocol (for example, a Bluetooth protocol) is utilized for data transfer between external physiological sensor devices 206 and IMD 202, while a different wireless data communication protocol (for example, a long range telemetry protocol) is utilized for data transfer between IMD 202 and telemetry transceiver device 208.

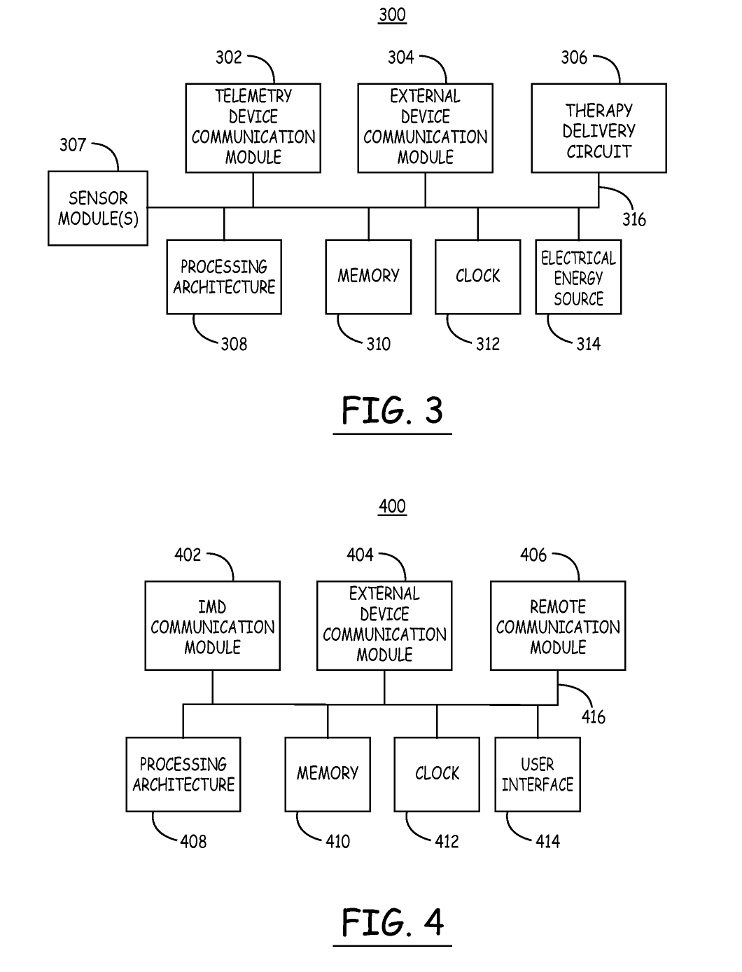

FIG. 3 is a schematic representation of an embodiment of an IMD 300 suitable for use in systems 100 and 200. Some of the features, functions, and characteristics of IMD 300 have already been described above. For the sake of brevity, such common features, functions, and characteristics will not be redundantly described here.

IMD 300 may include hardware, software, firmware, and circuitry for managing the operation and function of IMD 300, with such features being contained within a hermetic enclosure of IMD 300. IMD 300 includes a number of electrical components, operating modules, and components such as, without limitation: a telemetry device communication module 302; an external device communication module 304; a therapy delivery circuit 306; one or more sensor modules 307; a processing architecture 308; a suitable amount of memory 310, which may include random-access memory (RAM) and/or read-only memory (ROM); a clock 312; and an electrical energy source 314. These elements may be coupled together using, for example, a bus 316 or any suitably configured interconnection arrangement. Although not shown in FIG. 3, the communication modules 302/304 may cooperate with one or more antennas configured to enable IMD 300 to communicate with other devices.

Although not a requirement, this example assumes that IMD 300 is configured for cardiac applications (e.g., to provide cardiac sensing, pacing, and/or defibrillation functions for the patient). In certain embodiments, IMD 300 may include an implantable cardiac monitor without a therapy delivery function, e.g., an implantable ECG monitor for recording the cardiac electrogram from electrodes remote from the heart. Alternatively, IMD 300 may include an implantable hemodynamic monitor (IHM) for recording cardiac electrogram and other physiologic sensor derived signals, e.g., one or more of blood pressure, blood gases, temperature, electrical impedance of the heart and/or chest, heart sounds, and patient activity. In yet another embodiment, IMD 300 includes the combined functionality of sensing, pacing, and defibrillating.

IMD 300 may utilize endocardial electrode leads (not shown) to deliver electrical therapy to the patient's heart as needed. Accordingly, these leads are coupled to therapy delivery circuit 306. These leads may also be utilized to deliver sensor signals to IMD 300. Electrically, the coupling of the lead conductors to internal electrical components of IMD 300 may be facilitated by means of lead interface circuit (not shown). In practice, the lead interface circuit may function, in a multiplexer-like manner, to selectively and dynamically establish necessary connections between various conductors in the leads and individual electrical components of IMD 300, as would be familiar to those of ordinary skill in the art.

Sensor modules 307 are suitably configured to collect physiological data from the IMD sensors. Sensor modules 307 may include, for example, an ECG module for cardiac applications. Alternatively or additionally, sensor modules 307 may be configured to collect data from other sensor types such as sensors for monitoring fluid accumulation or cardiac performance parameters (stroke volume, pre-ejection interval, blood flow metrics and the like), pressure sensors, impedance sensing for respiratory parameters, acoustic sensors for heart sounds, accelerometers for patient activity, etc.

IMD 300 includes processing architecture 308, which generally varies in sophistication and complexity depending upon the type and functional features of IMD 300. In practice, one or more of the modules or components of IMD 300 shown in FIG. 3 (or any portion thereof) may be realized in or executed by processing architecture 308, memory 310, and/or elsewhere in IMD 300. In certain embodiments, processing architecture 308 can be an off-the-shelf programmable microprocessor, a microcontroller, a custom integrated circuit, or any of a wide variety of other implementations generally known. Although specific connections between processing architecture 308 and other components of IMD 300 are not shown in FIG. 3, it will be apparent to those of ordinary skill in the art that processing architecture 308, in conjunction with clock 312, functions to control the timed operation of telemetry device communication module 302, external device communication module 304, and therapy delivery circuitry 306. In certain embodiments, the functioning of processing architecture 308 would be under the control of firmware or programmed software algorithms stored in memory 310 (e.g., RAM, ROM, PROM and/or reprogrammable ROM), which are carried out using a processing unit of a typical microprocessor core architecture. In certain embodiments, processing architecture 308 can also include a watchdog circuit, a DMA controller, a lock mover/reader, a CRC calculator, and other specific logic circuitry coupled together by on-chip bus, address bus, and power, clock, and control signal lines in paths or trees in a manner well known in the art.

In certain embodiments, as is known in the art, electrical energy source 314 powers IMD 300 and can also be used to power electromechanical devices, such as valves or pumps, of a substance delivery IMD. Moreover, electrical energy source 314 may also be utilized to provide electrical stimulation energy of an ICD pulse generator, cardiac pacing pulse (IPG) generator, or other electrical stimulation and sensing generator as needed. In one preferred embodiment, IMD 300 is suitably configured to recharge electrical energy source 314 by electromagnetic coupling with an external apparatus using inductive and propagation coupling techniques. In practice, electrical energy source 314 may be coupled to a power supply circuit having power-on-reset (POR) capability. The power supply circuit can provide one or more low voltage power supply signals, the POR signal, one or more voltage reference sources, current sources, an elective replacement indicator (ERI) signal, etc. For the sake of clarity in the example block diagram provided in FIG. 3, the connections between electrical energy source 314 and the electrical components of IMD 300 are not shown, as one skilled in the art would be familiar with such connections.

In certain embodiments, processing architecture 308 and/or therapy delivery circuit 306 can be configured to process physiological signals that are used to trigger or modulate therapy delivery and are stored as physiological signal data for later retrieval. As mentioned above, IMD 300 may receive and process physiological sensor data that originates at one or more external physiological sensor devices, where such sensor data can influence the operation or activation of IMD 300. In a typical application, electrical signal sense electrodes and/or physiologic sensors are realized on or in the implanted electrode leads, and such sensors are situated at a site distanced from IMD 300. Alternatively (or additionally), sense electrodes may be realized on or in the housing of IMD 300. Alternatively (or additionally), sense electrodes may be connected to IMD 300 via feedthrough elements that traverse the housing of IMD 300 and may be in near proximity of the device or more remote using a wired connection (e.g., leads, tails and the like) or wireless (using sensors elsewhere in the body that communicate with the IMD through dedicated in-body telemetry).

In certain embodiments, the electrode leads are utilized to carry sensor signals to IMD 300, which can then process such signals to derive event signals reflecting the occurrence of specific cardiac electrical events, including atrial contractions (P-waves) and ventricular contractions (R-waves). These event-indicating signals are provided to processing architecture 308 for use in controlling the synchronous stimulating operations of IMD 300 in accordance with common practice in the art. In addition, these event indicating signals may be communicated, via uplink transmission, to a telemetry transceiver device (using telemetry device communication module 302), to an external physiological sensor device (using external device communication module 304), or to other external communication devices.

Telemetry device communication module 302 may be suitably configured to generate and receive RF signals that are transmitted and received by IMD 300 to support wireless data communication with a telemetry transceiver device. Communication module 302 may include or cooperate with one or more antennas (not shown). Communication module 302 may include or cooperate with any number of transmitters, any number of receivers, and/or any number of transceivers, depending upon the particular implementation. For example, communication module 302 may cooperate with a suitably configured far field telemetry transceiver to enable IMD 300 to perform telemetry communication with an external telemetry transceiver device in accordance with one or more of the wireless data communication protocols mentioned above.

External device communication module 304 may be suitably configured to generate and receive RF signals that are transmitted and received by IMD 300 to support wireless data communication with external physiological sensor devices (see FIG. 2). Communication module 304 may include or cooperate with one or more antennas (not shown). Communication module 304 may include or cooperate with any number of transmitters, any number of receivers, and/or any number of transceivers, depending upon the particular implementation. For example, communication module 304 may cooperate with a suitably configured wireless radio to enable IMD 300 to perform telemetry communication with an external physiological sensor devices in accordance with one or more of the wireless data communication protocols mentioned above.

In example embodiments, therapy delivery circuitry 306 can be configured to control and regulate the delivery of electrical stimulation to the patient, e.g., cardioversion/defibrillation therapy pulses and/or cardiac pacing pulses delivered to the heart, or other electrical stimulation delivered to the brain, other organs, selected nerves, the spinal column, the cochlea, or muscle groups, including skeletal muscle wrapped about the heart. Alternatively, in certain embodiments, therapy delivery circuitry 306 can be configured as a drug pump for delivering drugs into organs for therapeutic treatment or into the spinal column for pain relief. Alternatively, in certain embodiments, therapy delivery circuitry 306 can be configured to operate an implantable heart assist device or pump implanted in patients awaiting a heart transplant operation.

Registers of memory 310 can be used for storing data compiled from sensed cardiac activity and/or relating to device operating history or sensed physiologic parameters. Generally, the data storage can be triggered manually by the patient, on a periodic basis, in response to a control instruction received by the telemetry transceiver device, or by detection logic upon satisfaction of certain programmed-in event detection criteria. If not manually triggered, in certain embodiments, the criteria for triggering data storage within IMD 300 can be programmed via telemetry transmitted instructions and parameter values. Following such triggering, in certain embodiments, event related data, e.g., the date and time, may be stored along with the stored periodically collected or patient initiated physiologic data. Typically, once stored, the data is ready for telemetry transmission to the telemetry transceiver device on receipt of a retrieval or interrogation instruction.

Memory 310 may also be used to store data necessary to support the functionality of the system. For example, memory 310 may be configured to store one or more device identifiers, where each device identifier identifies one wireless device (such as an external physiological sensor device or the telemetry transceiver device) in the system. In practice, each device identifier is unique throughout at least the particular system. Indeed, each device identifier may be unique on a global scale or on any suitable scale beyond that of the system. These device identifiers can be employed for device synchronization, data packet acknowledgement, and the like.

FIG. 4 is a schematic representation of an embodiment of a telemetry transceiver device 400 suitable for use in systems 100 and 200. Some of the features, functions, and characteristics of telemetry transceiver device 400 have already been described above. For the sake of brevity, such common features, functions, and characteristics will not be redundantly described here.

Telemetry transceiver device 400 may include hardware, software, firmware, and circuitry for managing the operation and function of telemetry transceiver device 400. Telemetry transceiver device 400 includes a number of electrical components, operating modules, and components such as, without limitation: an IMD communication module 402; an external device communication module 404; a remote communication module 406; a processing architecture 408; a suitable amount of memory 410, which may include random-access memory (RAM) and/or read-only memory (ROM); a clock 412; and a user interface 414. In practice, some of these elements may be optional (for example, external device communication module 404 need not be utilized in system 200, and telemetry transceiver device 400 need not include any user interface 414). The elements of telemetry transceiver device 400 may be coupled together using, for example, a bus 416 or any suitably configured interconnection arrangement. Although not shown in FIG. 4, the communication modules 402/404/406 may cooperate with one or more antennas configured to enable telemetry transceiver device 400 to communicate with other devices.

IMD communication module 402 is suitably configured to wirelessly receive IMD data from one or more IMDs. IMD communication module 402 may also be configured to wirelessly transmit data, such as command and control instructions, to one or more IMDs. For an embodiment such as system 200, IMD communication module 402 may also be configured to wirelessly receive physiological sensor data from one or more IMDs, and to wirelessly transmit data, such as control instructions, status information, or alarm indicators, to one or more IMDs (for forwarding to external physiological sensor devices). In practice, IMD communication module 402 may include or cooperate with any number of transmitters, any number of receivers, and/or any number of transceivers, depending upon the particular implementation. For example, IMD communication module 402 may cooperate with a suitably configured RF transceiver to enable telemetry transceiver device 400 to perform telemetry communication with IMDs in accordance with one or more of the wireless data communication protocols mentioned above.

External device communication module 404 is suitably configured to receive (preferably via a wireless link) physiological sensor data from one or more external physiological sensor devices. External device communication module 404 may also be configured to wirelessly send data to one or more external physiological sensor devices (see FIG. 1). Such data may include, without limitation: command instructions, control instructions, status information, alarm indicators, reminders, or the like. In practice, external device communication module 404 may include or cooperate with any number of transmitters, any number of receivers, and/or any number of transceivers, depending upon the particular implementation. For example, external device communication module 404 may cooperate with a suitably configured RF transceiver to enable telemetry transceiver device 400 to perform telemetry communication with external physiological sensor devices in accordance with one or more of the wireless data communication protocols mentioned above.

Remote communication module 406 is suitably configured to send patient data to at least one remote computing architecture, where the patient data conveys the IMD data and/or the physiological sensor data. Remote communication module 406 may also be configured to receive data from one or more remote computing architectures. Such received data may represent control instructions (for an IMD, for an external physiological sensor device, and/or for telemetry transceiver device 400 itself) generated by the remote computing architecture, where such control instructions may be influenced by the patient data processed by the remote computing architecture. Alternatively or additionally, such received data may represent status information (for an IMD, for an external physiological sensor device, and/or for telemetry transceiver device 400 itself) generated by the remote computing architecture, where such status information may be influenced by the patient data processed by the remote computing architecture.

In practice, remote communication module 406 may be configured to support wired and/or wireless data communication with the remote computing architecture. For example, remote communication module 406 may be realized as a cellular telephone transceiver that communicates with a cellular carrier network. Alternatively, remote communication module 406 may be realized as an Ethernet or wireless network interface that is configured to communicate with a network such as a LAN, a WAN, the Internet, or the like. Therefore, remote communication module 406 may include or cooperate with any number of transmitters, any number of receivers, any number of transceivers, and any number of connection jacks, ports, or physical interfaces, depending upon the particular implementation and depending upon the supported wired and/or wireless data communication protocols.

Processing architecture 408 may vary in sophistication and complexity depending upon the type and functional features of telemetry transceiver device 400. In practice, one or more of the modules or components of telemetry transceiver device 400 shown in FIG. 4 (or any portion thereof) may be realized in or executed by processing architecture 408, memory 410, and/or elsewhere in telemetry transceiver device 400. In certain embodiments, processing architecture 408 can be an off-the-shelf programmable microprocessor, a microcontroller, a custom integrated circuit, or any of a wide variety of other implementations generally known. Although specific connections between processing architecture 408 and other components of telemetry transceiver device 400 are not shown in FIG. 4, it will be apparent to those of ordinary skill in the art that processing architecture 408, in conjunction with clock 412, functions to control the timed operation of the communication modules and other operations of telemetry transceiver device 400. In certain embodiments, the functioning of processing architecture 408 would be under the control of firmware or programmed software algorithms stored in memory 410 (e.g., RAM, ROM, PROM and/or reprogrammable ROM), which are carried out using a processing unit of a typical microprocessor core architecture.

User interface 414 may include any number of elements, components, or features that enable the patient, a caregiver, or any user of telemetry transceiver device 400 to interact with telemetry transceiver device 400. In this regard, user interface 414 may include, without limitation: switches, buttons, a keyboard or keypad, a display element, a touch screen, a mouse or other computer pointing device, speakers, a microphone, indicator lights, or the like. The user interface 414 can also be configured to prompt queries to the patient, e.g., in case the remote architecture (clinical decision support system) would require data on possible patient symptoms or other information from the patient.

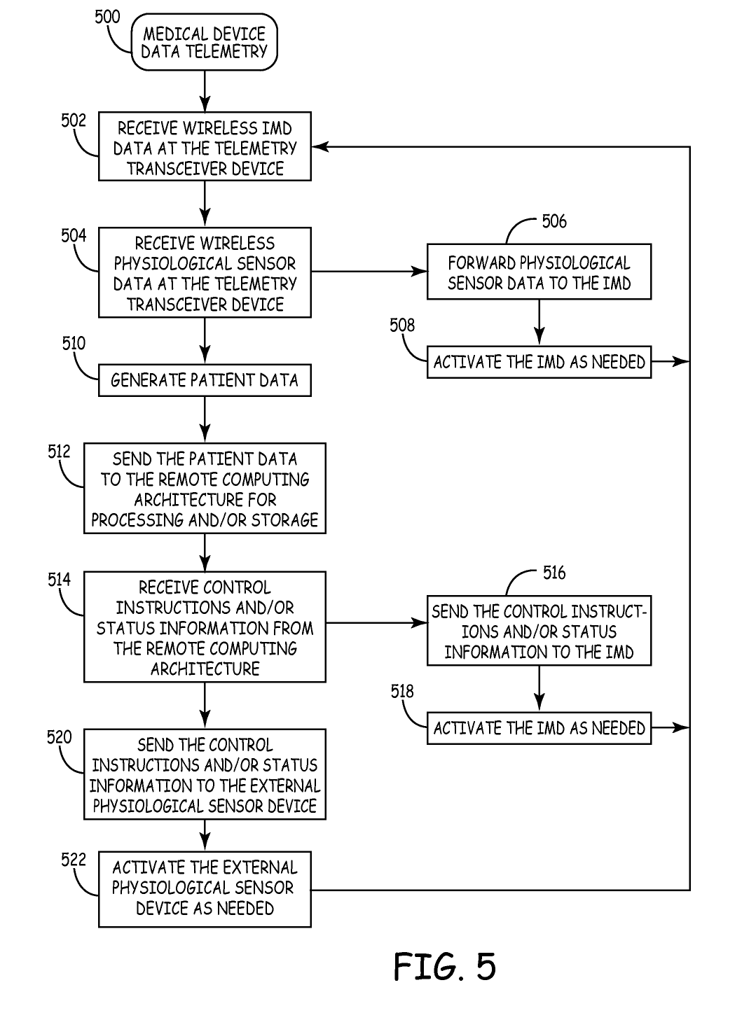

FIG. 5 is a flow chart that illustrates an embodiment of a medical device data telemetry process 500. The various tasks performed in connection with process 500 may be performed by software, hardware, firmware, or any combination thereof. For illustrative purposes, the following description of process 500 may refer to elements mentioned above in connection with FIGS. 1-4. In practice, portions of process 500 may be performed by different elements of the described system, e.g., an IMD, a telemetry transceiver device, an external physiological sensor device, or a remote computing architecture. It should be appreciated that process 500 may include any number of additional or alternative tasks, the tasks shown in FIG. 5 need not be performed in the illustrated order, and process 500 may be incorporated into a more comprehensive procedure or process having additional functionality not described in detail herein.

Medical device data telemetry process 500 may continuously or periodically monitor the system for IMD data and physiological sensor data. This way, a baseline of the various IMD data and external sensor data is established. In this regard, process 500 may wirelessly receive IMD data at the telemetry transceiver device (task 502) in accordance with a designated wireless data communication protocol, which will be referred to here as the “first” wireless data communication protocol. As explained above, the IMD data originates at an IMD in the system. The telemetry transceiver device may also obtain physiological sensor data generated by an external physiological sensor device. As described above, in one system embodiment, the external physiological sensor device wirelessly sends the physiological sensor data to the IMD, and the IMD wirelessly forwards the physiological sensor data to the telemetry transceiver device. For the preferred embodiment illustrated in FIG. 5, however, the telemetry transceiver device wirelessly receives the physiological sensor data directly from the external physiological sensor device (task 504). In practice, the physiological sensor data may be transferred in compliance with the first wireless data communication protocol or in compliance with a different wireless data communication protocol, which will be referred to here as the “second” wireless data communication protocol.

FIG. 5 depicts an optional task 506 during which the telemetry transceiver device wirelessly forwards the received physiological sensor data to the IMD. In one practical embodiment, the physiological sensor data is forwarded to the IMD in accordance with the first wireless data communication protocol. Thereafter, the IMD may process the physiological sensor data as needed, and medical device data telemetry process 500 may activate or operate the IMD in response to the physiological sensor data (task 508). In other words, the IMD may be suitably configured to react to the forwarded physiological sensor data (in addition to any physiological or other data collected by the IMD itself).

The telemetry transceiver device may collect, store, and process the received IMD data and the received physiological sensor data in any suitable manner. For example, the telemetry transceiver device may generate patient data that conveys the IMD data and/or the physiological sensor data (task 510). The patient data may be formatted and arranged as desired (e.g., as data packets). This patient data can then be sent to at least one remote computing architecture for processing, handling, formatting, and/or storage (task 512). In practice, the patient data is transferred in accordance with a wired or wireless network data communication protocol, which may be compliant with any of the data communication protocols described above (including the first wireless data communication protocol or the second wireless data communication protocol). In preferred embodiments, the patient data is transferred using a data protocol that is different than the first and second wireless data communication protocols. This data communication protocol will be referred to here as the “third” data communication protocol.

In response to the patient data, one or more applications running at the remote computing architecture may generate control instructions, status information, reminders, alarm or alert indicators, or other data intended for the IMD, the telemetry transceiver device, and/or for the external physiological sensor device. Data or instructions generated by the remote computing architecture can be transmitted back to the telemetry transceiver device using the third data communication protocol. The telemetry transceiver device receives the control instructions and/or the status information (task 514) and processes the received information as needed. In this regard, the telemetry transceiver device may send the control instructions and/or the status information to the IMD (task 516) using, for example, the first wireless data communication protocol. Thereafter, the IMD may be activated or operated as needed in response to the control instructions or the status information (task 518).

Alternatively or additionally, the telemetry transceiver device may send the control instructions and/or the status information to one or more external physiological sensor devices (task 520) using, for example, the second wireless data communication protocol. Thereafter, the external physiological sensor device may be activated or operated as needed in response to the control instructions or the status information (task 522).

FIG. 5 depicts medical device data telemetry process 500 as a continuous loop. This is intended to represent a practical implementation that supports 24-hour monitoring by the remote computing architecture.

While at least one example embodiment has been presented in the foregoing detailed description, it should be appreciated that a vast number of variations exist. It should also be appreciated that the example embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

1. A system for handling medical device data, the system comprising: an external physiological sensor device having wireless data communication capabilities;
a telemetry transceiver device configured to wirelessly receive implantable medical device (IMD) data from an IMD, and configured to obtain physiological sensor data generated by the external physiological sensor device;
a communication module for the telemetry transceiver device, the communication module being configured to send patient data that conveys the IMD data and the physiological sensor data; and
a remote computing architecture in data communication with the communication module, the remote computing architecture being configured to receive the patient data from the communication module.

2. A system according to claim 1, wherein the telemetry transceiver device is configured to wirelessly receive the physiological sensor data from the external physiological sensor device.

3. A system according to claim 2, wherein:
the telemetry transceiver device is configured to wirelessly receive the IMD data from the IMD in accordance with a first wireless data communication protocol; and
the telemetry transceiver device is configured to wirelessly receive the physiological sensor data from the external physiological sensor device in accordance with a second wireless data communication protocol that is different than the first wireless data communication protocol.

4. A system according to claim 3, wherein the second wireless data communication protocol is selected from the group consisting of:
an IrDA protocol, a Bluetooth protocol, an IEEE 802.15 protocol, an IEEE 802.11 protocol, an IEEE 802.16 protocol, a direct sequence spread spectrum protocol; a frequency hopping spread spectrum protocol, a cellular telephony protocol, a cordless telephony protocol, a wireless home network communication protocol, a paging network protocol, a magnetic induction protocol, a wireless medical telemetry service (WMTS) protocol, an industrial, scientific, and medical (ISM) protocol, a GPRS protocol, and a wireless USB protocol.

5. A system according to claim 2, wherein the telemetry transceiver device is configured to wirelessly forward the physiological sensor data to the IMD.

6. A system according to claim 1, wherein:
the external physiological sensor device is configured to wirelessly transmit the physiological sensor data to the IMD; and
the telemetry transceiver device is configured to wirelessly receive the physiological sensor data from the IMD.

7. A system according to claim 6, wherein:
the external physiological sensor device is configured to wirelessly transmit the physiological sensor data to the IMD in accordance with a wireless data communication protocol;
the telemetry transceiver device is configured to wirelessly receive the IMD data from the IMD in accordance with the wireless data communication protocol; and
the telemetry transceiver device is configured to wirelessly receive the physiological sensor data from the IMD in accordance with the wireless data communication protocol.

8. A system according to claim 6, wherein:
the external physiological sensor device is configured to wirelessly transmit the physiological sensor data to the IMD in accordance with a first wireless data communication protocol;
the telemetry transceiver device is configured to wirelessly receive the IMD data from the IMD in accordance with a second wireless data communication protocol that is different than the first wireless data communication protocol; and
the telemetry transceiver device is configured to wirelessly receive the physiological sensor data from the IMD in accordance with the second wireless data communication protocol.

9. A system according to claim 1, wherein the communication module is integrated into the telemetry transceiver device.

10. A system according to claim 9, wherein: the telemetry transceiver device is implemented as a mobile telephone; and the communication module comprises a mobile telephone transceiver configured to communicate the patient data via a mobile telephone network.

11. A system according to claim 9, wherein:
the telemetry transceiver device is implemented as a computing device having network connectivity; and
the communication module comprises a network interface configured to communicate the patient data via a network.

12. A system according to claim 11, wherein the network includes the Internet.

13. A system according to claim 1, wherein the external physiological sensor device is selected from the group consisting of:
a blood pressure measurement device, a body weight measurement device, a body fat measurement device, a body temperature measurement device, a blood glucose measurement device, a heart rate measurement device, and a lung capacity measurement device.

14. A system according to claim 1, wherein the remote computing architecture is configured to:
generate, in response to the patient data, control instructions for the external physiological sensor device; and
send the control instructions to the external physiological sensor device via the telemetry transceiver device.

15. A system according to claim 1, wherein the remote computing architecture is configured to:
generate, in response to the patient data, status information for the external physiological sensor device; and
send the status information to the external physiological sensor device via the telemetry transceiver device.

16. A system according to claim 15, wherein the status information comprises an alarm indication.

17. A telemetry transceiver device for handling medical device data, the telemetry transceiver device comprising:
an implantable medical device (IMD) communication module configured to wirelessly receive IMD data from an IMD;
an external device communication module configured to receive physiological sensor data from an external physiological sensor device; and
a remote communication module coupled to the IMD communication module and coupled to the external device communication module, the remote communication module being configured to send patient data to a remote computing architecture, wherein the patient data conveys the IMD data and the physiological sensor data.

18. A telemetry transceiver device according to claim 17, wherein:
the IMD communication module is configured to wirelessly receive the IMD data from the IMD in accordance with a first wireless data communication protocol; and
the external device communication module is configured to wirelessly receive the physiological sensor data from the external physiological sensor device in accordance with a second wireless data communication protocol that is different than the first wireless data communication protocol.

19. A telemetry transceiver device according to claim 17, wherein the remote communication module comprises a mobile telephone transceiver configured to send the patient data to a mobile telephone network.

20. A telemetry transceiver device according to claim 17, wherein the remote communication module comprises a network interface configured to send the patient data to a data communication network.

21. A telemetry transceiver device according to claim 17, wherein:
the remote communication module is configured to receive control instructions from the remote computing architecture, the control instructions being influenced by the patient data; and
the external device communication module is configured to send the control instructions to the external physiological sensor device.

22. A telemetry transceiver device according to claim 17, wherein:
the remote communication module is configured to receive status information for the external physiological sensor device, the status information being influenced by the patient data; and
the external device communication module is configured to send the status information to the external physiological sensor device.

23. A method for handling medical device data, the method comprising:
wirelessly receiving implantable medical device (IMD) data at a telemetry transceiver device, the IMD data originating at an IMD;
the telemetry transceiver device obtaining physiological sensor data generated by an external physiological sensor device;
the telemetry transceiver device generating patient data that conveys the IMD data and the physiological sensor data; and
the telemetry transceiver device sending the patient data to a remote computing architecture.

24. A method according to claim 23, wherein obtaining the physiological sensor data comprises wirelessly receiving the physiological sensor data from the external physiological sensor device.

25. A method according to claim 24, wherein:
wirelessly receiving the IMD data is compliant with a first wireless data communication protocol; and
wirelessly receiving the physiological sensor data is compliant with a second wireless data communication protocol that is different than the first wireless data communication protocol.

26. A method according to claim 24, further comprising the telemetry transceiver device wirelessly forwarding the physiological sensor data to the IMD.

27. A method according to claim 26, further comprising activating the IMD in response to the physiological sensor data.

28. A method according to claim 23, further comprising the external physiological sensor device wirelessly sending the physiological sensor data to the IMD, wherein obtaining the physiological sensor data comprises wirelessly receiving the physiological sensor data from the IMD.

29. A method according to claim 28, wherein:
wirelessly sending the physiological sensor data is compliant with a wireless data communication protocol;
wirelessly receiving the IMD data is compliant with the wireless data communication protocol; and
wirelessly receiving the physiological sensor data is compliant with the wireless data communication protocol.

30. A method according to claim 28, wherein:
wirelessly sending the physiological sensor data is compliant with a first wireless data communication protocol;
wirelessly receiving the IMD data is compliant with a second wireless data communication protocol that is different than the first wireless data communication protocol; and
wirelessly receiving the physiological sensor data is compliant with the second wireless data communication protocol.

31. A method according to claim 28, further comprising activating the IMD in response to the physiological sensor data.

32. A method according to claim 23, further comprising:
the telemetry transceiver device receiving control instructions from the remote computing architecture, the control instructions being influenced by the patient data; and
the telemetry transceiver device sending the control instructions to the external physiological sensor device.

33. A method according to claim 23, further comprising:
the telemetry transceiver device receiving status information from the remote computing architecture, the status information being influenced by the patient data; and
the telemetry transceiver device sending the status information to the external physiological sensor device.

34. A method according to claim 23, further comprising processing the patient data at the remote computing architecture.

35. A method according to claim 23, further comprising storing the patient data at the remote computing architecture.
